(19) United States
(12) Patent Application Publication
(10) Pub. No.: US 2012/0269802 A1
(43) Pub. Date: Oct. 25, 2012

(54) Treatment And Prognosis With Thalidomide In Multiple Myeloma Based on Karyotyping And Gene Expression Profiling

(75) Inventors: John D. Shaughnessy, JR., Roland, AR (US); Bart Barlogie, Little Rock, AR (US)

(73) Assignee: Board Of Trustees Of The University Of Arkansas

(21) Appl. No.: 13/501,933

(22) PCT Filed: Oct. 13, 2010
(86) PCT No.: PCT/US2010/002734

§ 371 Date: Jul. 2, 2012

Related U.S. Application Data
(60) Provisional application No. 61/278,878, filed on Oct. 13, 2009.

(51) Int. Cl. A61K 39/395; A61K 31/203; C40B 30/04; A61K 38/30; A61K 31/7088; A61P 35/00; C12Q 1/68; A61K 38/18

(52) U.S. Cl. 424/133.1; 435/6.12; 514/559; 514/7.6; 424/174.1; 514/8.7; 514/44 A; 506/9

(57) ABSTRACT

The present invention provides a method treating a myeloma patient by administering one or more of thalidomide, a Total Therapy 2 regimen, an interleukin-6 signaling suppressor, an interleukin-6R signaling suppressor, an IGF1 signaling suppressor, an IGF1 R signaling suppressor, shRNA or other modulators of gene expression. Also, provided are methods for predicting outcome of a treatment for an individual having a cancer, e.g., myeloma, by performing one or more of karyotyping or expression profiling of chromosomes 1 and 13 or expression level measurement of IL-6R.

### CROSS-REFERENCE TO RELATED APPLICATION

This international application claims benefit of priority under 35 U.S.C. §119(e) of provisional application U.S. Ser. No. 61/278,878, filed Oct. 13, 2009, now abandoned, the entirety of which is hereby incorporated by reference.

### FEDERAL FUNDING LEGEND

This invention was supported in part by National Institutes of Health No: CA55819. Consequently, the federal government has certain rights in this invention.

### BACKGROUND OF THE INVENTION

1. Field of the Invention

The present invention generally relates to the field of cancer research. More specifically, the present invention relates to predicting the outcome of treatments in multiple myeloma patients and potential therapeutic effects of thalidomide in individuals with certain cytogenetic abnormalities linked in elevated expression of IL6R and IGF1R. By utilizing gene expression profiling, myeloma patients may know ahead of time, the likely outcomes to specific therapeutic regimens including whether or not thalidomide would be beneficial.

2. Description of the Related Art

Multiple myeloma is an invariantly fatal B cell malignancy that manifests at the plasma cell stage of differentiation. Although multiple myeloma initially resides in the bone marrow, it can transform into an aggressive disease with increased proliferation (resulting in a higher frequency of abnormal metaphase karyotypes), elevated LDH and extramedullary manifestations. Additionally, the clinical course of multiple myeloma and its response to therapy is influenced by special molecular genetic lesions and tumor cell-microenvironment interaction.

Although complete response can be obtained in more than 40% of patients with high-dose therapy, survival can vary from few months to more than fifteen years. Furthermore, high-risk disease is best captured by abnormal metaphase cytogenetics, present in 30% to 50% of newly diagnosed patients and reflecting a higher proliferative capacity and stromal cell-independence of the malignant clone. However, karyotypes of multiple myeloma are notoriously complex and have until recently defied cytogenetic classification. Nevertheless, a comprehensive correlative analyses of multiple myeloma karyotypes with patient survival from multiple laboratories now reveal that hyperdiploid, non-hyperdiploid, chromosome 13 deletion-positive, t(4;14)(p16;q32)-positive, and t(11;14)(q13;q32)-positive forms of the disease likely represent unique subclasses with divergent clinical outcomes.

There is also evidence that multiple myeloma is characterized by chromosome 1 instability at the cytogenetic level. Chromosome 1 instability generally involves partial duplications, whole-arm translocations or jumping translocations of 1q identified by G-banding. This instability was further characterized recently using a combination of spectral karyotyping and fluorescence in situ hybridization (FISH) with probes for satII/III (1q12), BCL9 (1q21), and IL6R (1q21) on the karyotypes of 44 patients with known 1q aberrations. In eight patients, segmental duplication of 1q12-21 and adjacent bands occurred on non-homologous chromosomes. In five cases, the 1q first jumped to a non-homologous chromosome, after which the 1q12-21 segment subsequently again duplicated itself one to three times. In three other cases, segmental duplications occurred after the 1q first jumped to a non-homologous chromosome and then duplicated the adjacent proximal non-homologous chromosome segment prior to jumping or inserting to a new location. These cases demonstrate that satII/III DNA sequences are not only associated with duplication of adjacent distal chromosome segments after translocation, but are also associated with duplication and jumping/insertion of proximal non-homologous chromosome segments. There is also evidence that deletion of chromosome 13 in multiple myeloma is associated with upregulation of the IGF1R gene mapping to chromosome 15. Tumor cells of the MF subtype are known to express high levels of IGF1 and IGF1R. This group of patients also tend to overexpress IL6R gene.

While the presence of an abnormal karyotype has emerged as a significant prognostic variable in predicting outcome in patients receiving high dose chemotherapy and tandem stem cell transplants, this variable in combination with other historically relevant clinical parameters, e.g. serum albumin, b2M, and lactate dehydrogenase, account for no more than 30% of the variability in outcome in this disease. Thus, there is a need for more robust risk stratification algorithms for this disease.

Furthermore, the survival impact of new agents, such as bortezomib and thalidomide and its derivatives, will be profound if their clinical efficacy also extends to genetically defined high-risk myeloma, which has not been investigated.

A frustrating aspect of cancer chemotherapy is the unpredictable variability of induction or duration of response and long-term survival. In particular, in myeloma patients, a significant percentage (approximately 20%) derive no tangible benefit from the therapy, but still are subjected to drug toxicity, secondary risk, reduced quality of life, and delay in treatment that might have been effective.

The prior art is thus deficient in providing a method of predicting the outcomes to specific therapeutic regimens including whether or not thalidomide would be beneficial. The present invention fulfills this long-standing need and desire in the art.

### SUMMARY OF THE INVENTION

The present invention is directed to a method of treating an individual with multiple myeloma comprising administering a pharmacologically effective amount of thalidomide to the individual, thereby treating the multiple myeloma.

The present invention is directed to a related method further comprising administering pharmacologically effective amounts of the drugs in Table 1 according to a Total Therapy 2 regimen. The present invention is directed to another related method further comprising administering a pharmacologically effective amount of a compound that inhibits interleukin 6 signaling. The present invention is directed to another related method further comprising administering a pharmacologically effective amount of a compound that suppresses signaling through interleukin-6R. The present invention is directed to another related method further comprising administering a pharmacologically effective amount of a compound that suppresses IGF1 signaling. The present invention is directed to another related method further comprising administering a pharmacologically effective amount of shRNA or other modulators of gene expression.

The present invention is directed to another related method further comprising predicting an outcome of the treatment by obtaining plasma cells from the individual and karyotyping chromosomes 1 and 13, where the presence of an anomaly in either chromosomes but not both indicates favorable outcome. The present invention is directed to another related method further comprising predicting an outcome of the treatment by performing gene expression profiling on chromosomes 1 and 13 to determine low-risk or high-risk myeloma. The present invention is directed to another related method further comprising predicting an outcome of the treatment by measuring expression levels of interleukin-6R by myeloma cells, wherein high expression levels indicates poor outcome of treatment.

The present invention is further directed to a method predicting outcome of treatment for an individual having a cancer. The method comprises obtaining plasma cells from the individual and karyotyping chromosomes 1 and 13, wherein the presence of an anomaly in either chromosomes but not in both indicates favorable prognosis. The present invention is directed to a related method further comprising performing gene expression profiling on chromosomes 1 and 13 to determine whether the multiple myeloma is low-risk or high-risk. The present invention is directed to another related method further comprising measuring expression levels of IL6R, wherein high expression levels indicates poor outcome of treatment.

The present invention is directed further to a method for predicting outcome of treatment for an individual having multiple myeloma. The method comprises obtaining plasma cells from the individual and performing one or more analyses on the plasma cells. The analyses comprise a karyotype on chromosomes 1 and 13, where the presence of an anomaly in either chromosome but not both indicates favorable outcome, gene expression profiling on chromosomes 1 and 13 to determine whether the myeloma is a low-risk or high-risk multiple myeloma; wherein the high-risk multiple myeloma is determined by over-expression of genes on chromosome 1q, under-expression of genes on chromosome 1p or reduced expression of genes on chromosome 13q and the low risk myeloma is GEP defined or expression level measurement of IL6R, wherein high expression levels indicates poor outcome of treatment.

Other and further aspects, features, and advantages of the present invention will be apparent from the description of the presently preferred embodiments of the invention. These embodiments are given for the purpose of disclosure.

### BRIEF DESCRIPTION OF THE DRAWINGS

FIGS. 1A-1C show Kaplan-Meier survival plots of patients receiving Total Therapy 2 according to randomization to the control arm (Thal−) or to thalidomide (Thal+) and the presence of cytogenetic abnormalities (CA).

FIG. 2 shows a histogram plot of gene expression profiling (GEP)-defined risk scores according to cytogenetic abnormalities subgroup designations (CA1 plus [CA1+13], CA1 but not CA13 but not CA1, cytogenetic abnormalities other than CA1 and CA13).

FIG. 3 shows a boxplot depicting Affymetrix-based GEP values of the IL6R gene, according to cytogenetic subgroups.

### DETAILED DESCRIPTION OF THE INVENTION

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “or” in the claims refers to “and/or” unless explicitly indicated to refer to alternatives only or the alternatives are mutually exclusive, although the disclosure supports a definition that refers to only alternatives and “and/or”.

In one embodiment of the present invention, there is provided a method for treating multiple myeloma in an individual, comprising administering to the individual a pharmacologically effective amount of thalidomide, thereby treating the cancer.

In a further embodiment the method comprises administering pharmacologically effective amounts of the drugs in Table 1 according to a Total Therapy 2 regimen.

In another further embodiment, the method comprises administering a pharmacologically effective amount of a compound that inhibits interleukin 6 signaling. In one aspect of this further embodiment, the compound may suppress interleukin-6. Examples of interleukin-6 suppressors are retinoic acid and Activin A. In another aspect, the compound may suppress one or more interleukin-6 activating factors or one or more factors upstream or downstream therefrom. In this aspect, the interleukin-6 activating factor is IL-1, TNF-a, STAT3, or JAK2. Particularly, the factor may be JAK2 and the compound is AG490. Also, the factor may be IL-1 and the compound is anti-IL1 antagonist. In addition the factor may be DKK1 and the compound is an anti-DKK1 antibody. In yet another aspect the compound may be a neutralizing antibody or other biological mediators of ligand receptor interaction.

In yet another further embodiment, the method comprises administering a pharmacologically effective amount of a compound that suppresses signaling through interleukin-6R. An example of interleukin-6R suppressor is tocilzumab.

In yet another further embodiment the method comprises administering a pharmacologically effective amount of a compound that suppresses IGF1 signaling. An example of an IGF1 signaling suppressor is IGFBP3.

In yet another further embodiment, the method comprises administering a pharmacologically effective amount of a compound that suppresses signaling through IGF1R.

In yet another further embodiment the method comprises administering a pharmacologically effective amount of shRNA or other modulators of gene expression.

In yet another further embodiment, the method comprises predicting an outcome of the treatment by obtaining plasma cells from the individual; and karyotyping chromosomes 1 and 13, wherein the presence of an anomaly in either chromosome but not both indicates favorable outcome. In this further embodiment the anomaly may be detected by interphase in situ fluorescent hybridization and/or metaphase in situ fluorescent hybridization.

Further to this embodiment the method comprises performing gene expression profiling on chromosomes 1 and 13 to determine low-risk or high-risk myeloma. In one aspect of this embodiment the high-risk multiple myeloma may be determined by over-expression of genes on chromosome 1q, under-expression of genes on chromosome 1p or reduced expression of genes on chromosome 13q. In another aspect of this embodiment, the myeloma may be GEP defined low risk multiple myeloma. In another further embodiment, the method comprises measuring expression levels of interleukin-6R by myeloma cells, wherein high expression levels indicates poor outcome of treatment.

In another embodiment of the present invention, there is provided a method for predicting an outcome of treatment for an individual having a cancer: comprising obtaining plasma cells from the individual; and karyotyping chromosomes 1 and 13, wherein the presence of an anomaly in either chromosomes but not in both indicates favorable prognosis. In a further embodiment, the method comprises measuring expression levels of IL6R, where high expression levels indicates a poor outcome of treatment.

In an aspect of these embodiments, the cancer is multiple myeloma where the method comprises performing gene expression profiling on chromosomes 1 and 13 to determine whether the multiple myeloma is low-risk or high-risk. In this aspect the high-risk multiple myeloma is determined by over-expression of genes on chromosome 1q, under-expression of genes on chromosome 1p or reduced expression of genes on chromosome 13q. Alternatively, the myeloma is gene expression profiling defined low risk multiple myeloma.

In all embodiments and aspects thereof the anomaly may be detected by interphase in situ fluorescent hybridization and/or metaphase in situ fluorescent hybridization. A representative cancer includes but is not limited to myeloma.

In yet another embodiment of the present invention, there is provided a method for predicting outcome of treatment for an individual having multiple myeloma, comprising obtaining plasma cells from the individual; and performing one or more analyses on the plasma cells comprising a karyotype on chromosomes 1 and 13, wherein the presence of an anomaly in either chromosome but not both indicates favorable outcome; gene expression profiling on chromosomes 1 and 13 to determine whether the myeloma is a low-risk or high-risk multiple myeloma; wherein the high-risk multiple myeloma is determined by over-expression of genes on chromosome 1q, under-expression of genes on chromosome 1p or reduced expression of genes on chromosome 13q and the low risk myeloma is gene expression profiling defined; or expression level measurement of IL6R, wherein high expression levels indicates poor outcome of treatment. In this embodiment the anomaly may be detected by interphase in situ fluorescent hybridization and/or metaphase in situ fluorescent hybridization.

Provided herein are methods of treating a cancer, for example, but not limited to, myeloma, in an individual. Treatment may comprise administration of a compound such as thalidomide with or without the therapeutic regimen Total Therapy 2 (TT2) described herein. Additional treatments may include the suppression of one or more of interleukin-6R signaling, such as with tocilizumab, IGF1 signaling, such as with IGFBP3, or IGF1R signaling or gene expression modulators, such as shRNA or other modulators.

It is contemplated that inhibition of signaling cascades induced by interleukin-6 activating factors or, alternatively, inhibition of factors upstream or downstream of IL-6 provides further therapeutic options for treating myeloma. Thus, additional treatment regimens may further include administration of one or more compounds effective to inhibit or suppress interleukin-6 signaling. As such, effective therapeutic compounds may suppress or inhibit interleukin-6, e.g., retinoic acid and Activin A. Effective therapeutic compounds may suppress or inhibit interleukin-6 activating factors, such as, IL-1, TNF-alpha or STAT3. For example, the compound AG490 suppresses JAK2, an anti-IL 1 antagonist inhibits interleukin-1, an anti-DKK1 antibody inhibits DKK1. Other representative inhibitor or suppressor compounds are neutralizing antibodies or other biological mediators of ligand receptor interactions.

The present invention also provides methods of predicting the outcome of these treatment regimens by karyotyping chromosomes, e.g., chromosomes 1 and 13, in plasma cells obtained from an individual undergoing treatment for myeloma. The presence of an anomaly in one, but not both, of the chromosomes is predictive of a favorable outcome. Types of cytogenetic abnormalities or anomalies include, but not limited to, deletions and/or duplications of part of one or more chromosomes. Abnormalities also can be of the entire chromosomes. As is known in the art, chromosome abnormalities or anomalies are detected by interphase in situ fluorescent hybridization and/or metaphase in situ fluorescent hybridization.

In addition, gene expression profiling of chromosomes 1 and 13 can differentiate between low risk and high risk myeloma. Particularly, compared to control, such as a healthy individual, over-expression of genes on chromosome 1q, under-expression of genes on chromosome 1p or reduced expression of genes on chromosome 13q are indicative of high risk myeloma. Alternatively, low risk myeloma is gene expression profiling defined. Furthermore, a high expression level of interleukin-6R on myeloma cells is indicative of a poor treatment outcome. As is standard and known in the art, altered gene expression may be detected by DNA microarray, PCR-based assays, protein-based assays or a combination.

Particularly, thalidomide in Total Therapy 2 (TT2) for myeloma benefited patients exhibiting cytogenetic abnormalities (CA). Table 1 shows the treatment details of Total Therapy 2. To clarify the underlying mechanism, survival was examined in the 351 patients for whom gene expression profiling and cytogenetic data were available. GEP-defined high-risk status was largely conferred by expression of genes residing on chromosomes 1 and 13. Survival in the context of cytogenetic abnormalities involving chromosomal loci (CA1, CA13) was also examined. Statistical methods including Cox regression modeling were employed to define variables independently impacting outcomes. While confirming superior survival with thalidomide only in patients with cytogenetic abnormalities-type myeloma, consideration of gene expression profiling-defined risk revealed that this benefit was restricted to the larger subset with low-risk disease.

In the context of CA1 and CA13, thalidomide's benefit in low-risk myeloma was limited to patients with CA1/13. The equally poor survival in case of CA1+13 was linked to significantly higher expression levels of IL6R, which was an independent adverse parameter for survival in addition to TP53 haplo-insufficiency, gene expression profiling high-risk, cytogenetic abnormalities, and high beta-2-microglobulin; thalidomide randomization was favorable. Thalidomide's survival benefit pertained to gene expression profiling low-risk myeloma exhibiting CA1/13 that, unlike CA1+13, was not associated with hyper-activation of IL6R.

TABLE 1
Treatment Details of Total Therapy 2

| | |
|---|---|
| INDUCTION | |
| Cycle 1 | Vincristine (0.5 mg/d × 4 d) continuous infusion |
| VAD | Adriamycin (10 mg/m2/d × 4 d) continuous infusion |
| | Dexamethasone 40 mg PO d 1-4, 9-12, 17-20 |
| Cycle 2 | Cyclophosphamide (400 mg/m2/day × 4 d) continuous infusion |
| DCEP | Etoposide (40 mg/m2/day × 4 d) continuous infusion |
| | cis-Platin (10 mg/m2/day × 4 d) continuous infusion |
| | Dexamethasone 40 mg/d) PO days 1-4 |
| Cycle 3 | Cyclophosphamide (750 mg/m2/day × 4 d) continuous infusion |
| CAD | Adriamycin (15 mg/m2/d × 4 d) continuous infusion |
| | G-CSF 10 μg/kg sq twice daily → PBSC (peripheral blood stem |
| | cell) collection |
| Cycle 4 | Cyclophosphamide (400 mg/m2/day × 4 d) continuous infusion |
| DCEP | Etoposide (40 mg/m2/day × 4 d) continuous infusion |
| | cis-Platin (10 mg/m2/day × 4 d) continuous infusion |
| | Dexamethasone (40 mg/d) PO days 1-4 |
| TRANSPLANT | |
| 1st Transplant | Melphalan 200 mg/m2 d −1 |
| | (reduced to 140 mg/m2 for: age >7 Cycle 5 0 years or creatinine |
| | >3 mg/dL) |
| 2nd Transplant | Melphalan 200 mg/m2 d−1 |
| | (reduced to 140 mg/m2 for: age >70 years or creatinine >3 mg/dL) |
| | < PR: |
| | BCNU 300 mg/m2 d−5 |
| | Etoposide 200 mg/m2 |
| | Arabinosyl Cytosine 400 mg/m2 d −5 through −2 |
| | Melphalan 140 mg/m2 d−2 |
| CONSOLIDATION | |
| Randomize | Cyclophosphamide (300 mg/m2/d × 4 d) continuous infusion |
| Arm A | Etoposide (30 mg/m2/day × 4 d) continuous infusion |
| | cis-Platin (7.5 mg/m2/day × 4 d) continuous infusion |
| | Dexamethasone 40 mg/d PO days 1-4 every 3 months for 4 cycles |
| Randomize | Alternating with |
| Arm B | Cyclophosphamide (400 mg/m2/day × 4 d) continuous infusion |
| | Adriamycin (10 mg/m2/day × 4 d) continuous infusion |
| | Dexamethasone 40 mg PO days 1-4 every 6 weeks for 8 cycles |
| PROTOCOL | after entry of 121 patients (66 DCEP, 55 DCEP/CAD) |
| MODIFICATION: | |
| DPACE | cis-Platin (7.5 mg/m2/day × 4 d) continuous infusion |
| | Adriamycin (7.5 mg/m2/day × 4 d) continuous infusion |
| | Cyclophosphamide (300 mg/m2/day × 4 d) continuous infusion |
| | Etoposide (30 mg/m2/day × 4 d) continuous infusion |
| | Dexamethasone 40 mg PO days 1-4 Q 3 months × 4 |
| DEX | In case of failure to recover platelets to at least 100,000/μL or |
| | failure to achieve >25% M-protein reduction with 1st cycle of |
| | DCEP in induction: DEX 40 mg d 1-4 q 28 d for 1 year. |
| MAINTENANCE | |
| Interferon | 3 million units/m2 SQ 3 x wk plus 1st year of maintenance: |
| | Dexamethasone 40 mg every 3 months on days 1-4, 9-12, 17-20 |
| | 2nd year onward: interferon alone |

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Total Therapy 2 Treatment in Myeloma Patients

Of all 668 patients accrued to TT2, the 351 with gene expression profiling data available on CD138-purified plasma cells prior to initiation of therapy are the subjects of this report. Their 6-year overall and event-free survival estimates of 63% and 42%, respectively, are similar to the 60% and 47% recorded for the 317 patients lacking gene expression profiling information. The gene expression profiling subgroup is considered representative of the overall TT2 population. The sole clinical outcome focus in this example is the overall survival measured from initiation of therapy.

All patients had signed a written informed consent in keeping with institutional and Food and Drug Administration guidelines and in accordance with the Helsinki Declaration. The protocols and their revisions had been approved by the Institutional Review Board, which also had reviewed and approved annual follow-up reports. A Data Monitoring Board reviewed outcome data annually. An independent, federally accredited investigator team audited TT2 charts semi-annually for protocol adherence, accuracy of outcome annotations and of toxicity entries in approximately 80% of cases enrolled.

Kaplan-Meier methods were used to generate survival distribution graphs and comparisons were made using the log-rank test. Categorical comparisons were made using the chi-squared test. Running log-rank tests were used to determine optimal cut points for IL6R expression levels. Multivariate analyses applied stepwise selection and Cox proportional hazard regression modeling.

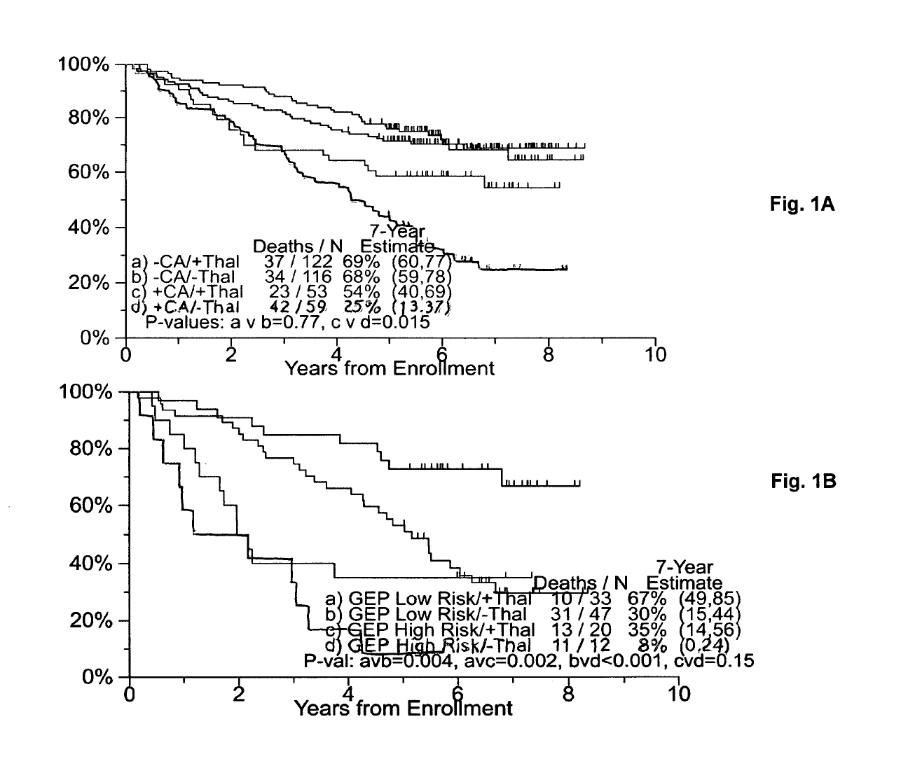

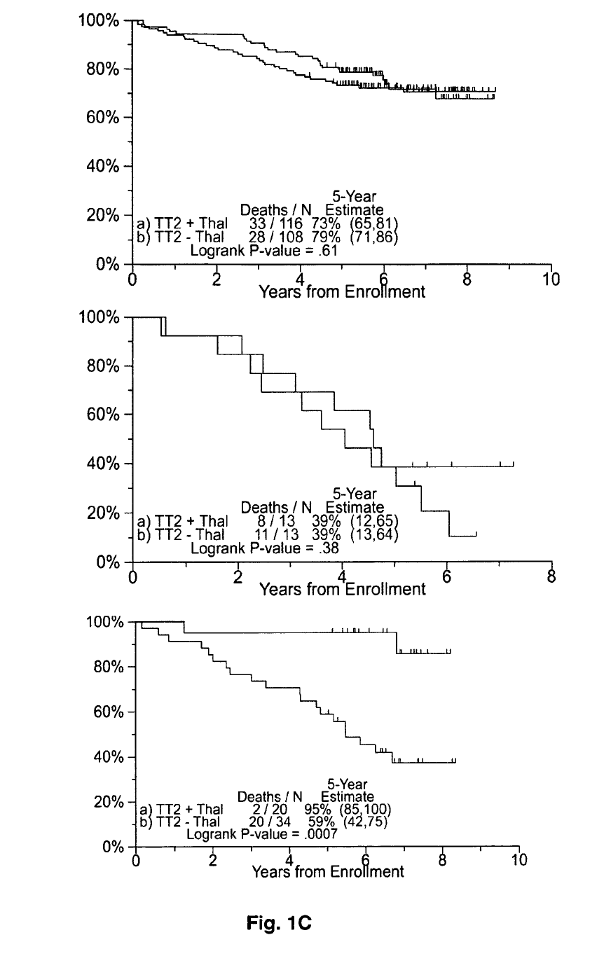

Relative to thalidomide's impact in TT2, we confirm that its benefit was restricted to patients exhibiting cytogenetic abnormalities, resulting in 7-year survival improvement from 25% for patients treated on the control arm to 54% for those randomized to thalidomide (p=0.015) (FIG. 1A). While only trending toward better outcomes in all patients with gene expression profiling-defined low risk myeloma (p=0.13), the 7-year survival estimate in the subset of patients displaying cytogenetic abnormalities was more than doubled to 67% on the thalidomide versus 30% on the control arm (p=0.004) (FIG. 1B).

Among the 70 genes distinguishing high-risk from low-risk myeloma, many reside on chromosomes 1 (CA1) and 13 (CA13). Adverse prognosis was linked to over-expression of genes residing on chromosome 1q, under-expression of those on chromosome 1p and reduced expression of genes on chromosome 13q, consistent with metaphase cytogenetic data reported previously. With this background, survival outcomes of low-risk patients treated on the two arms of TT2 were examined in the context of 3 cytogenetic abnormalities subgroups: no cytogenetic abnormalities; presence of both CA1 and CA13 abnormalities (CA1+13); and presence of cytogenetic abnormalities without both CA1 and CA13 (CA1/13). Survival outcomes on both treatment arms were similar in the patient subgroup exhibiting no cytogenetic abnormalities and in the prognostically unfavorable subgroup displaying CA1+13 (FIG. 1C), while, in the CA1/13 subgroup, the addition of thalidomide vastly improved 5-year survival estimates from 59% to 95% (p<0.001).

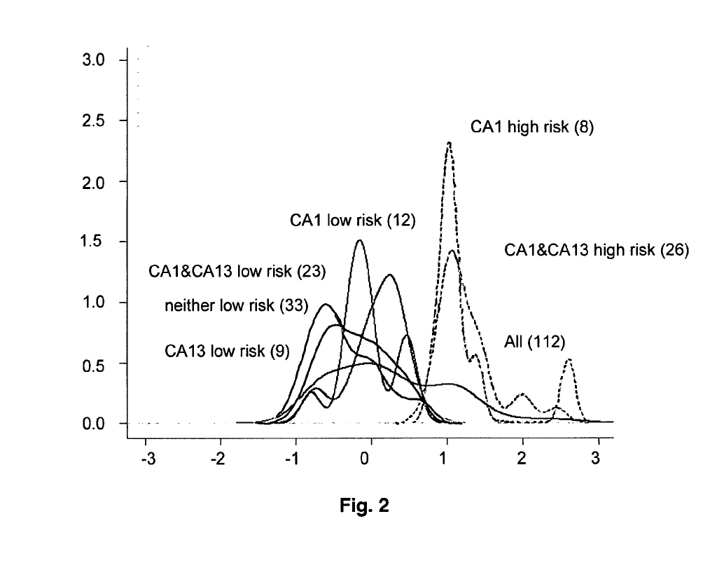

As the observed survival differences between cytogenetic abnormalities subgroups might be related to differences in gene expression profiling-derived risk scores, their distributions were examined according to CA1 and CA13 designations (FIG. 2). For all patients, risk scores spanned a wide range (−1.398 to +2.601; median, −0.173). In the low-risk category, lowest median and peak scores were observed among patients lacking cytogenetic abnormalities and those with only CA13. Bimodal distributions were present in case of CA1 and CA1+13. Those with CA1 exhibited dominance of the lower risk score mode while the converse applied to those with CA1+13. The high-risk group was devoid of no CA or only CA13 designations. Risk score distributions were similar among CA1 only and CA1+13 categories.

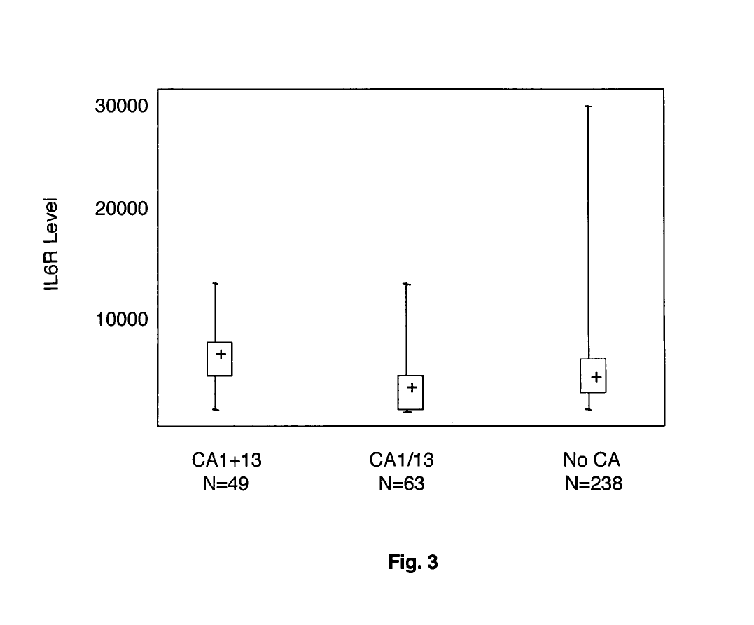

Searching for genes differentially expressed among no cytogenetic abnormalities, CA1/13 and CA1+13 groups, IL6R was significantly over-expressed only in the combined CA1+13 group, regardless of gene expression profiling-defined risk status (FIG. 3). Relative to molecular subgroup representations, MS and PR designations were significantly over-represented in the CA1+13 category and HY was under-represented (38%, 15%; 15%) compared to patients without CA (10%, 4%; 27%) and CA1/13 (6%, 6%; 50%) (p<0.001, p=0.030; p=0.001).

Three gene expression profiling-derived parameters (high-risk, TP53 deletion, and IL6R >=2900 as the optimal cut-point), the presence of cytogenetic abnormalities, and beta-2-microglobulin (B2M) levels exceeding 5.5 mg/L emerged as independent parameters adversely affecting survival of patients treated with TT2, while randomization to the thalidomide arm reduced the hazard of death (Table 2). Hyper-expression of the CKS1B gene, residing on chromosome 1q21, conferred short survival when examined on univariate analysis, which was not retained after adjusting for the other variables. Among the subset with low-risk myeloma, the same variables pertained, including high IL6R's adverse connotation displacing both CA1 and CA13 from the model (Table 3). Applying an interaction term between cytogenetic abnormalities and thalidomide, patients with cytogenetic abnormalities at baseline experienced improved survival when treated with thalidomide, yet cytogenetic abnormalities remained as an adverse variable overall.

In conclusion, the present invention has demonstrated that thalidomide's benefit in TT2 was limited to the gene expression profiling-defined low-risk group with co-existing cytogenetic abnormalities. Further consideration of cytogenetic abnormalities details revealed, compellingly, that this difference could be traced to the presence of metaphase abnormalities involving chromosome 1 or 13, but not both. While patients exhibiting no cytogenetic abnormalities fared equally well on both arms of TT2, thalidomide benefited those with cytogenetic abnormalities who did not have abnormalities on both chromosomes 1 and 13. Having abnormalities on both of these chromosomes was uniquely characterized by markedly higher IL6R expression levels on myeloma cells which, according to multivariate Cox analysis, displaced univariately significant CA1 and CA13 categories from the survival model. Further investigations are in progress to elucidate the basis for thalidomide's lack of benefit in the presence of high levels of IL6R expression by myeloma cells.

In each of Tables 2-3: HR—Hazard Ratio, 95% Cl-95% Confidence Interval, P-value from Wald Chi-Square Test in Cox Regression NS2-Multivariate results not statistically significant at 0.05 level. All univariate p-values reported regardless of significance. Multivariate model uses stepwise selection with entry level 0.1 and variable remains if meets the 0.05 level. A multivariate p-value greater than 0.05 indicates variable forced into model with significant variables chosen using stepwise selection. Variables considered for the multivariate model were: Cytogenetic abnormalities, CA1, CA13, CA 1+13, Albumin <3.5 g/dL, B2M >5.5 mg/L, Creatinine ≧2 mg/dL, Hb <10 g/dL, LDH ≧190 U/L, GEP high-risk, GEP TP53 deletion, GEP IL6R expression ≧2900, GEP CKS1B Q4, Randomization to thalidomide, IL6R and CA 1+13 (interaction term).

TABLE 2
Univariate and multivariate analyses of baseline parameters associated
with overall survival of all patients treated on Total Therapy 2 (with gene
expression profiling data)

| | | | Overall Survival | |
|---|---|---|---|---|
| Table 1: all patients with GEP data | | | | P- |
| | Variable | n/N (%) | HR (95% CI) | value |
| Univariate | Age ≧65 yr | 69/351 (20%) | 1.14 (0.75, 1.72) | 0.546 |
| Analysis | Cytogenetic | 112/350 (32%) | 2.34 (1.67, 3.27) | <.001 |
| | abnormalities | | | |
| | CA1 | 69/350 (20%) | 3.04 (2.14, 4.33) | <.001 |
| | CA13 | 59/350 (17%) | 2.79 (1.92, 4.04) | <.001 |
| | CA 1 + 13 | 49/350 (14%) | 3.49 (2.38, 5.11) | <.001 |
| | Albumin | 56/347 (16%) | 1.53 (1.00, 2.33) | 0.050 |
| | <3.5 g/dL | | | |
| | B2M ≧3.5 mg/L | 144/351 (41%) | 2.23 (1.59, 3.13) | <.001 |
| | B2M >5.5 mg/L | 72/351 (21%) | 2.33 (1.63, 3.35) | <.001 |
| | Creatinine | 37/342 (11%) | 2.54 (1.64, 3.93) | <.001 |
| | ≧2 mg/dL | | | |
| | CRP ≧4 mg/L | 184/348 (53%) | 1.13 (0.81, 1.59) | 0.468 |
| | CRP ≧8 mg/L | 124/348 (36%) | 1.17 (0.83, 1.66) | 0.365 |
| | Hb <10 g/dL | 98/351 (28%) | 1.51 (1.07, 2.14) | 0.020 |
| | LDH ≧190 U/L | 118/351 (34%) | 2.00 (1.42, 2.80) | <.001 |
| | GEP high-risk | 46/351 (13%) | 4.01 (2.71, 5.95) | <.001 |
| | GEP TP53 | 35/351 (10%) | 2.65 (1.70, 4.12) | <.001 |
| | deletion | | | |
| | GEP IL6R | 194/351 (55%) | 1.90 (1.33, 2.71) | <.001 |
| | expression ≧2900 | | | |
| | (optimal cut) | | | |
| | GEP CKS1B Q4 | 88/351 (25%) | 1.86 (1.30, 2.67) | <.001 |
| | Randomization to | 175/351 (50%) | 0.78 (0.56, 1.10) | 0.158 |
| | thalidomide | | | |
| Multi- | Cytogenetic | 110/337 (33%) | 1.95 (1.36, 2.78) | <.001 |
| variate | abnormalities | | | |
| Analysis | B2M >5.5 mg/L | 69/337 (20%) | 1.73 (1.18, 2.52) | 0.005 |
| | GEP high-risk | 45/337 (13%) | 2.68 (1.75, 4.11) | <.001 |
| | GEP TP53 | 34/337 (10%) | 3.09 (1.97, 4.85) | <.001 |
| | deletion | | | |
| | GEP IL6R | 188/337 (56%) | 1.68 (1.15, 2.44) | 0.007 |
| | expression ≧2900 | | | |
| | (optimal cut) | | | |
| | Randomization to | 168/337 (50%) | 0.69 (0.49, 0.98) | 0.037 |
| | thalidomide | | | |

TABLE 3
Univariate and multivariate analyses of baseline parameters associated
with overall survival of low-risk patients treated on Total Therapy 2 (with
gene expression profiling data)

| Table 2: patients with | | | OS from Enrollment | |
|---|---|---|---|---|
| GEP-defined low-risk | | | | P- |
| | Variable | n/N (%) | HR (95% CI) | value |
| Univariate | Age ≧65 yr | 60/305 (20%) | 1.34 (0.84, 2.13) | 0.218 |
| Analysis | Cytogenetic | 80/304 (26%) | 2.05 (1.38, 3.05) | <.001 |
| | abnormalities | | | |
| | CA1 | 38/304 (13%) | 2.68 (1.71, 4.22) | <.001 |
| | CA13 | 35/304 (12%) | 2.33 (1.44, 3.77) | <.001 |
| | CA 1 + 13 | 26/304 (9%) | 3.28 (1.99, 5.42) | <.001 |
| | Albumin | 40/301 (13%) | 1.48 (0.88, 2.49) | 0.143 |
| | <3.5 g/dL | | | |
| | B2M ≧3.5 mg/L | 117/305 (38%) | 2.38 (1.61, 3.52) | <.001 |
| | B2M >5.5 mg/L | 55/305 (18%) | 2.68 (1.76, 4.08) | <.001 |
| | Creatinine | 28/297 (9%) | 3.04 (1.82, 5.08) | <.001 |
| | ≧2 mg/dL | | | |
| | CRP ≧4 mg/L | 156/303 (51%) | 1.10 (0.74, 1.62) | 0.643 |
| | CRP ≧8 mg/L | 105/303 (35%) | 1.19 (0.80, 1.77) | 0.395 |
| | Hb <10 g/dL | 82/305 (27%) | 2.11 (1.42, 3.12) | <.001 |
| | LDH ≧190 U/L | 91/305 (30%) | 1.69 (1.13, 2.53) | 0.010 |
| | GEP TP53 | 29/305 (10%) | 2.75 (1.65, 4.57) | <.001 |
| | deletion | | | |
| | GEP IL6R | 159/305 (52%) | 1.94 (1.29, 2.91) | 0.001 |
| | expression ≧2900 | | | |
| | (optimal cut) | | | |
| | GEP CKS1B Q4 | 51/305 (17%) | 1.14 (0.69, 1.91) | 0.604 |
| | Randomization to | 149/305 (49%) | 0.74 (0.50, 1.10) | 0.140 |
| | thalidomide | | | |
| Multi- | Randomization to | 149/304 (49%) | 1.08 (0.65, 1.79) | 0.766 |
| variate | thalidomide | | | |
| Analysis | Cytogenetic | 80/304 (26%) | 3.17 (1.89, 5.30) | <.001 |
| | abnormalities | | | |
| | CA with | 33/304 (11%) | 0.27 (0.11, 0.64) | 0.003 |
| | thalidomide | | | |
| | randomization | | | |
| | (interaction term) | | | |
| | B2M >5.5 mg/L | 55/304 (18%) | 2.57 (1.68, 3.95) | <.001 |
| | TP53 deletion | 29/304 (10%) | 3.13 (1.86, 5.25) | <.001 |
| | IL6R | 159/304 (52%) | 1.89 (1.26, 2.86) | 0.002 |
| | Expression | | | |
| | ≧2900 | | | |
| | (optimal cut) | | | |

The following references are cited herein.
1. Barlogie et al., Blood. 2008; 112:3115-3121.
2. Shaughnessy et al., Blood. 2007; 109: 2276-2284.
3. Zhan et al., Blood. 2006; 108:2020-2028.
4. Barlogie et al., Brit J Haematol. 2007; 138: 176-85.
5. Xiong et al., Blood. 2008; 112:4235-4246.
6. Kaplan et al., J Am Stat Assoc. 1958; 53: 457-48.
7. Gooley et al., Stat Med. 1999; 18:695-706.
8. Crowley et al., In: Lecture Notes in Statistics. Proceedings of the First Seattle Symposium in Biostatistics: Survival Analysis. Seattle, Wash. 1997; 199-229.
9. Cox DR. Regression models and life-tables. J R Stat Soc [B] 1972; 34: 187-202.
10. Jacobson et al., Brit J Haemat. 2003; 122: 430-440.
11. Desikan et al., Blood. 2000; 95: 4008-4010.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

1-33. (canceled)

34. A method of identifying a multiple myeloma patient as being responsive to a treatment regimen comprising thalidomide, comprising testing the level of gene expression of interleukin-6 receptor (IL-6R) in plasma cells isolated from a multiple myeloma patient determined to have gene expression profiling (GEP)-defined low-risk multiple myeloma, wherein the presence of a normal IL-6R gene expression level, relative to a suitable control, indicates that the subject will be responsive to a treatment regimen comprising thalidomide.

35. A method of treating a subject having multiple myeloma comprising administering a treatment regimen comprising a therapeutically effective amount of thalidomide to the subject, wherein the subject was previously determined to be responsive to the treatment by the method of claim 34 and the treatment regimen is administered to the subject on the basis of the determination.

36. A method of predicting the presence of a cytogenetic abnormality on both chromosome 1 and chromosome 13 in a subject having multiple myeloma, comprising testing the level of gene expression of IL6-R in plasma cells isolated from the subject, wherein an elevated IL6-R gene expression level, relative to a suitable control, indicates that the subject has a cytogenetic abnormality on both chromosome 1 and chromosome 13.

37. A method of treating a subject having multiple myeloma comprising administering a treatment regimen without thalidomide to the subject, wherein the subject was previously determined to have a cytogenetic abnormality on both Chromosome 1 and Chromosome 13 by the method of claim 36 and the treatment regimen is administered to the subject on the basis of the determination.

38. The method of claim 37, wherein the treatment regimen comprises a therapeutically effective amount of a compound that suppresses interleukin-6 activity.

39. The method of claim 38, wherein the compound is retinoic acid or Activin A.

40. The method of claim 38, wherein the compound suppresses one or more interleukin-6 activating factors or one or more factors upstream or downstream therefrom.

41. The method of claim 40, wherein the interleukin-6 activating factor is IL-1, TNF-a, STAT3, or JAK2.

42. The method of claim 41, wherein the factor is JAK2 and the compound is AG490.

43. The method of claim 41, wherein the factor is IL-1 and the compound is anti-IL1 antagonist.

44. The method of claim 40, wherein the factor is DKK1 and the compound is an anti-DKK1 antibody.

45. The method of claim 38, wherein the compound is a neutralizing antibody.

46. The method of claim 37, wherein the treatment regimen comprises a therapeutically effective amount of a compound that suppresses signaling through interleukin-6R.

47. The method of claim 46, wherein the compound is tocilizumab.

48. The method of claim 37, wherein the treatment regimen comprises a therapeutically effective amount of a compound that suppresses IGF1 signaling.

49. The method of claim 48, wherein the compound is IGFBP3.

50. The method of claim 37, wherein the treatment regimen comprises a therapeutically effective amount of a compound that that suppresses signaling through IGF1R.

51. The method of claim 37, wherein the treatment regimen comprises a therapeutically effective amount of shRNA.

52. The method of claim 34, further comprising testing the presence of GEP-defined TP53 deletion in the subject.

53. The method of claim 36, further comprising testing the presence of GEP-defined TP53 deletion in the subject.
